(19) United States
(12) Patent Application Publication
(10) Pub. No.: US 2009/0155381 A1
(43) Pub. Date: Jun. 18, 2009

(54) Method of treatment or prevention of age-related macular degeneration

(76) Inventors: Regina Goralczyc, Grenzach-Wyhlen (DE); Antoine De Saizieu, Brunstatt (FR); Wolfgang Schalch, Bottmingen (DE); Igor Bendik, Oberwil (CH)

Correspondence Address: NIXON & VANDERHYE, PC, 901 NORTH GLEBE ROAD, 11TH FLOOR, ARLINGTON, VA, 22203, US

(21) Appl. No.: 11/991,400

(22) PCT Filed: Sep. 8, 2006
(86) PCT No.: PCT/GB2006/003331

§ 371 Date: Jun. 3, 2008

(30) Foreign Application Priority Data
Sep. 8, 2005 (EP) 05019532.0

(51) Int. Cl. A61K 33/34; A61P 27/02

(52) U.S. Cl. 424/638

(57) ABSTRACT

A method of treatment and/or prevention of age-related macular degeneration (AMD) wherein, in a first step, the need for treatment or susceptibility to AMD is determined for an individual and, in a second step, a medication comprising lutein and/or zeaxanthin and/or certain antioxidants (or a mixture thereof) is tailored to that individual. The invention also provides a method of determining a substance to be administered to an individual, which individual may be susceptible of having age-related macular degeneration (or an age-related macular degeneration-related disorder) comprising: a) determining the susceptibility of the individual to age-related macular degeneration (usually genetically, by detection of an SNP); and b) on the basis of the determination in a), identifying a substance capable of preventing or treating age-related macular degeneration in that individual. The method may additionally comprise providing (such as administering or communicating) the substance (or its identity) to the individual.

### FIELD OF THE INVENTION

The present invention relates to a novel method for the (treatment and/or) prevention of age related macular degeneration (AMD). It relates to the diagnosis and/or treatment of age-related macular degeneration (or an age-related macular degeneration-related disorder) in an individual (or subject) by determining susceptibility of the individual to age-related macular degeneration and, on the basis of that determination, selecting or identifying (and administering) a substance to the individual.

### BACKGROUND OF THE INVENTION

As the most common cause of vision loss among people over the age of 60, macular degeneration impacts millions of older adults every year. The disease affects central vision and can sometimes make it difficult to read, drive or perform other activities requiring fine, detailed vision. When the macula is damaged, the eye loses its ability to see detail, such as small print, facial features or small objects. The damaged parts of the macula often cause scotomas, or localized areas of vision loss.

There are two types of the disease: dry macular degeneration and wet macular degeneration. Ninety percent of people who have macular degeneration have the dry form of the condition. In dry macular degeneration or atrophic macular degeneration, waste products may accumulate in the tissues underneath the macula forming yellowish deposits called drusen. The continued presence of drusen interferes with the blood flow to the retina and, in particular, to the macula. Less blood flow reduces the nourishment to the macula causing its light sensitive cells to stop working efficiently, or atrophy.

With wet macular degeneration, new weak blood vessels may grow in or under the retina causing fluid and blood to leak into the space under the macula. As a result, wet macular degeneration is sometimes called exudative macular degeneration, or described as choroidal neovascularization. The choroid is the area of blood vessels beneath the retina, and neovascularization refers to growth of new blood vessels in tissue. In choroidal neovascularization, blood vessels from the choroid grow into the macula.

The most common early sign of dry macular degeneration is blurred vision. As fewer cells in the macula are able to function, people will see details less clearly in front of them, such as faces or words in a book. If the loss of these light-sensing cells becomes great, people may see a small—but growing—blind spot in the middle of their vision.

The classic early symptom of wet macular degeneration is that straight lines appear crooked. This occurs when fluid from the leaking blood vessels gathers and lifts the macula, distorting vision. A small blind spot may also appear in wet macular degeneration, resulting in loss of one's central vision.

Regular eye exams are necessary for early detection of macular degeneration since symptoms may or may not be present in people who have the disease. Early drusen can be seen in an eye exam before symptoms develop.

The disease typically develops over an extended period of time and becomes apparent mostly not before it has reached an advanced stage. Further, while the etiology so far has remained largely unclear it has been reported that susceptibility for AMD may inter alia be genetically predetermined, see Science, Vol. 308 (2005), p. 419-424.

The present invention relates to the identification of those subjects which can have increased risk for developing (dry and/or wet) AMD and with the treatment and, particularly, prevention of AMD in those subjects suitably at an early as possible point in time.

### SUMMARY OF THE INVENTION

The present invention thus relates to a method for treatment and/or prevention of age-related macular degeneration (AMD) which comprises:

(a) identifying the individual risk of a subject (or individual, the terms are used interchangeably), of developing AMD or suffering from AMD; and

(b) providing an effective amount of a (preferably macular) carotenoid, in particular a xanthophyll, such as lutein and/or zeaxanthin and/or vitamin C, vitamin E; beta carotene, zinc and/or copper, and/or or a mixture thereof (the AREDS Cocktail, as described later) to said subject.

The present invention also provides a method of determining a substance to be administered to an individual, which individual may be susceptible of having age-related macular degeneration AMD, (which term includes a (wet or dry) age-related macular degeneration-related disorder or condition unless otherwise specified), the method comprising:

a) determining the susceptibility of the individual to age-related macular degeneration (AMD); and

b) on the basis of the determination in (a), identifying or selecting a substance capable of preventing and/or treating age-related macular degeneration (AMD) in that individual.

The method may additionally comprise:

c) providing (such as administering or communicating) the substance (or its identity) to the individual.

The invention may provide a method of treatment and/or prevention of age-related macular degeneration, which method comprises:

a) identifying or determining the risk of an individual developing age-related macular degeneration, or suffering from age-related macular degeneration; and

b) providing an effective amount of the substance to the individual (such as a human or animal, but usually the former), where the substance may be able to prevent or treat age-related macular degeneration (which terminology includes ameliorating or mitigating symptoms of age-related macular degeneration).

The invention further provides:

i) means capable of detecting an SNP or allelic variant related or associated with age-related macular degeneration in an individual and means for providing (such as administering or communicating) a substance (or its identity) capable of preventing or treating age-related macular degeneration to the individual;
ii) a kit for carrying out the method of the invention comprising means for detecting an SNP or allelic variant and an effective amount of (a (preferably macular) carotenoid, in particular a xanthophyll, such as lutein and/or zeaxanthin and/or vitamin C, vitamin E; beta carotene, zinc and/or copper, and/or or a mixture thereof (the AREDS Cocktail);
iii) a method of preparing a customised or personalised composition for an individual which is susceptible to age-related macular degeneration (or an age-related macular degeneration-related disorder), the method comprising:

(a) determining whether the individual is susceptible to age-related macular degeneration, or age-related macular degeneration-related disorder by a method of the invention; and

(b) preparing a composition suitable for, or tailored to, the individual;

iv) a method of providing a customised composition, comprising providing a composition suitable for a subject which is susceptible to age-related macular degeneration (or an age-related macular degeneration-related disorder), wherein the individual has been (eg. genetically) determined to be susceptible to age-related macular degeneration (or an age-related macular degeneration-related disorder);
v) a method of identifying a substance for the treatment of age-related macular degeneration (or an age-related macular degeneration-related disorder), the method comprising:

(a) contacting an age-related macular degeneration allelic variant polypeptide or a polynucleotide which encodes an age-related macular degeneration allelic variant with a test agent; and

(b) determining whether the substance is capable of binding to the polypeptide or modulating the activity or expression of the polypeptide or polynucleotide, and providing (such as administering or communicating) the substance (or its identity) to an individual;

vi) use of a compound which is therapeutic for age-related macular degeneration (or a age-related macular degeneration-related disorder) in the manufacture of a medicament for the prevention or treatment of age-related macular degeneration (or an age-related macular degeneration-related disorder) in a individual that has been identified as being susceptible to age-related macular degeneration by a method of the invention;
vii) a method of treating an individual for age-related macular degeneration (or an age-related macular degeneration-related disorder), the method comprising administering to the individual an (effective amount of a) therapeutic substance or compound which can prevent or treat AMD or a related disorder, wherein the individual has been identified as being susceptible to age-related macular degeneration, or an age-related macular degeneration-related disorder, by a method of the invention;
viii) a database comprising information relating to age-related macular degeneration allelic variants and optionally their association with age-related macular degeneration related disorder(s) and/or substances capable of preventing or treating age-related macular degeneration;
ix) a method for determining whether an individual is susceptible to age-related macular degeneration, or an age-related macular degeneration-related disorder, the method comprising:

(a) inputting data of one or more allelic variant(s) present in the subject to a computer system;

(b) comparing the data to a computer database, which database comprises information relating to age-related macular degeneration allelic variants and the age-related macular degeneration related disorder susceptibility associated with the variants; and

(c) determining on the basis of the comparison whether the individual is susceptible to age-related macular degeneration a related disorder;

x) a computer program comprising program code means that, when executed on a computer system, instructs the computer system to perform a method according to the invention;
xi) a computer program product comprising a computer-readable storage medium having recorded thereon a computer program according to the invention;
xii) a computer program product comprising program code means on a carrier wave, which program code means, when executed on a computer system, instructs the computer system to perform a method according to the invention;
xiii) a computer system arranged to perform a method according to the invention comprising:

(a) means for receiving data of the one or more age-related macular degeneration allelic variant(s) present in the individual;

(b) a module for comparing the data with a database comprising information relating to age-related macular degeneration allelic variants and the age-related macular degeneration related disorder susceptibility associated with the variants; and

(c) means for determining, on the basis of said comparison whether the individual is susceptible to age-related macular degeneration (or an age-related macular degeneration-related disorder);

xiii) a method of preparing a customised composition for an individual which is susceptible to age-related macular degeneration (or an age-related macular degeneration-related disorder), the method comprising:

(a) determining whether the individual is susceptible to age-related macular degeneration (or a age-related macular degeneration-related disorder) by a method of the invention;

(b) determining (such as electronically generating) a customised composition suitable for the individual;

(c) optionally, generating (e.g. electronic) manufacturing instructions to control the operation of composition manufacturing apparatus in accordance with the customised composition formulation; and

(d) manufacturing the customised food (according to the manufacturing instructions); or

xiv) use of a computer system of the invention to make a customised composition product.

### DETAILED DESCRIPTION OF THE INVENTION

According to the first aspect of the present invention, there is provided a method of determining a substance to be administered to an individual (or subject, the terms are used interchangeably), the method comprising:

a) determining the susceptibility of the individual to age-related macular degeneration (or an age-related macular degeneration-related disorder);

b) on the basis of the determination in a), identifying a substance capable of preventing and/or treating age-related macular degeneration in that individual.

The method may additionally comprise:

c) providing (e.g. administering or communicating) the substance (or its identity) to the individual (or subject).

Step c) can thus comprise communicating the identity of that substance to the individual, for example proposing, suggesting or recommending that substance. For instance, this may involve supplementing a person's food or diet with said substance.

### Determination of Susceptibility

The individual may be susceptible to age-related macular degeneration, or an age-related macular degeneration-related disorder. This may mean that they are at risk of, or have a predisposition to, age-related macular degeneration or an age-related macular degeneration-related disorder. That individual may not, in fact, necessarily have an increased risk or susceptibility, depending on the determination. The determination may find that the person has an increased risk, or (on the contrary) a decreased risk, of said disorder.

The determining stage in (a) may comprise preparing or obtaining a pharmacogenomic and/or nutritional profile (or identity) of the individual. This may assist in determining the susceptibility (such as risk or predisposition) to a disorder.

Thus, the determination may comprise:

i) conducting or performing a genome or genetic analysis of the individual;
ii) preparing a pharmacogenomic or metabolomic profile and/or identity based on personal and/or clinical information from or about the individual;
iii) performing a test or assay (such as on a biological sample from the individual) for a marker (e.g a biomarker, such as a compound present in the individual's body) or a physical condition that can indicate susceptibility (to age-related macular degeneration).

In (i), this may involve determining an individual's genotype. It can comprise identifying an allelic variant, a polymorphism (such as an SNP) or genetic predisposition (to the relevant disorder). One may therefore be able to draw up a genetic profile of the individual, preferably relevant to the disorder.

The determination may, alternatively or in addition to (ii), comprise obtaining relevant information from, or about, that individual. That information may be personal and/or clinical information. The information may relate, directly or indirectly, to age-related macular degeneration (or an age-related macular degeneration-related disorder). Such information may comprise information concerning lifestyle, health, nutritional status, diet. Other personal information of relevance can include age, sex, weight and/or ethnic background. Clinical information may comprise current drug and/or vitamin regimes, current or past treatments, familial data, health risks, family background, medical conditions and/or allergies. It may therefore involve obtaining a patient's history, and determining their nutritional profile. The individual may be able to provide this information, for example, by completing a questionnaire.

### Determining Risk/Susceptibility

### (i) Genetics

In one embodiment of the present invention the identification of the individual risk of a subject of developing AMD, or suffering from AMD (which may have been undetected so far) can be accomplished by genetic (or genome) analysis, more specifically, by determining the presence of gene polymorphism involved in (the development of) AMD.

Such a polymorphism may be present in the gene coding for the Complement Factor H, e.g., caused by sequence (SNP) rs1061170, known as coding variant Y402H (see Haines L., et al., Science 2005, 308:419, and Edwards A. O., et al., Science 2005, 308:421).

Further genes and/or polymorphisms (eg. SNPs) that may be tested for, or that may lead to development of AMD, can be:

coding for the angiotensin-converting enzyme, eg. where the Alu repeat is lacking (Hamdi et al, BBRC 295(3):668-672, 2002);

coding for the short-chain collagen CTRP-5, e.g. with changes at the Ser163Arg position (Hayward et al., Human Molecular Genetics 12(20):2657-2667, 2003);

coding for ABCR, e.g. having mutations ABCA4 and/or ABCA1 (Shroyer et al., Human Molecular Genetics 10(23:2671-2678, 2001);

coding for paraoxonase (PON) e.g. when carrying the genotype Gln192Arg and/or Met54Leu (Ikea et al, American J Opthalmology 132(2):191-195, 2001);

coding for an apolipoprotein E (APOE) variant, e.g. when carrying the epsilon-2 allele or lacking the epsilon 4-allele (Schmidt S., et al., Ophthalmic Genet. 2002, 23 (4), 209-223); Baird P N., et al., IOVS. 45(5):1311-1315, 2004);

coding for pigment-epithelium-derived factor (PEDF), e.g. when carrying the allele Met72Thr in exon 3 (Yamagishi S., et al., Med. Hypotheses 64(6):1202-4, 2005);

coding for CX3CR1, which encodes the fractalkine (chemokine, CX3CL1) receptor, e.g. and has one or two single nucleotide polymorphisms (SNPs): V2491 and T280M (Chan C C., et al., Histol Histopathol. 2005, 20(3):857-63;

coding for toll-like receptor 4 variant D299G (Zareparsi S., et al., Hum Mol Genet. 2005, 14(11):1449-55);

coding for Hepatic Lipase −514C->T and/or hepatic lipase −480C—>T polymorphism (Zhang C., et al., Am J Clin Nutr 2005, 81:1429-35; Bos G., et al., Am J Clin Nutr 2005, 81: 911-5);

coding for vascular endothelial growth factor (VEGF), e.g. polymorphism in the VEGF upstream promoter/leader sequence determined by three SNPs −2578C/A, −1154G/A and −1154G/G, and −634G/C (previously denoted +405C/G, position relative to the transcription start site) as well as −634C/C and in the VEGF 5′-untranslated region polymorphism −460C/T (Terry, P D. Et al., J Neurogenet 2004, 18(2):429-34; Howell W M., et al., J Med. Genet. 2005, 42(6):485-90; Awata T., et al., BBA 2005, 333:679-685; Koukourakis M I., et al., Lung Cancer 2004, 46(3):293-8; Hoon K., et al., Hum Reprod, 2005, epub ahead of print.). VEGF is involved in AMD pathology and promotes choroidal neovascularisation;

coding for MMP-9 microsatellite (CA (13-17)) polymorphism, e.g. variants that have > or 22 CA repeats (Fiotti N., et al., Genet. Med. 2005, 7(4):272-7). MMP9 is involved in choroidal neovascularisation (Lambert V., et al., Am J Pathol 2002, 161(4):1247-1253); and

coding for mutations in the tissue inhibitor of metalloproteinases-3 (TIMP3), such as the 3′ splice site mutation in the TIMP3 gene, namely a single base insertion at the intron 4/exon 5 junction which converts the consensus sequence CAG to CAAG in the splice acceptor site (Tabata Y Hum Genet. 1998 August; 103(2): 179-82), or a mutant TIMP3 allele, S181C TIMP3 or E139×TIMP3 (Arris C E et al., Biochim Biophys Acta. 2003; 1638(1):20-8.

The presence of polymorphism in genes encoding proteins which are related to the development of AMD can be determined by methods known in the art.

In general, this involves the extraction of genomic DNA by standard procedures (Sambrook J, Fritsch E F, and Maniatis T.: Molecular Cloning: A Laboratory Manual. Cold Spring Harbour Press; 1989) from blood cells, or buccal mucosa cells, hair cells or any other DNS containing human tissues which are easy in usually non-invasively accessible. Alternatively, commercial kits can be used (i.e. QIAMP blood kit, Qiagen or any other commercially available DNA extraction kit).

The characterization of a given genotype, which relates to the determination of the variants or polymorphisms, can be performed according to standard procedures. These classic technologies (Cotton R G H, Mutation detection, Oxford, Oxford University Press, 1997) that relate to the determination of the polymorphisms include DNA sequencing using 96-channel capillary sequencers, single-strand conformation analysis using non-denaturing gel electrophoresis, denaturing gradient gel electrophoresis using the partial melting behaviour of double-stranded DNA, heteroduplex analysis involving denaturing high-performance liquid chromatography, chemical or enzymatic cleavage of mismatch pairing method, and mutation detection by coupled transcription-translation (protein truncation test) procedures involving non-sense stop codons. The more recent techniques are real-time PCR methods like TaqMan, mass spectrometry involving single-stranded PCR fragments generated by the dideoxy-nucleotide PCR methodology, DNA MicroArray technology detecting SNPs provide by commercial suppliers (e.g. Affymetrix Inc., Santa Clara, Calif.) and many more. The identification of the haplotypes for the specific genes listed follows the procedures in the respective references cited for each gene.

### (ii) Optically

In a further embodiment of the invention, the determination or identification of the individual risk or susceptibility of a subject of developing AMD or suffering from AMD can be accomplished optically, such as by measuring optical density, e.g. of a macular pigment or other suitable optical measurement in or of the eye (e.g the retina). This can comprise measuring the level of a carotenoid, usually a macular carotenoid (such as lutein or zeaxanthin) in the eye of the individual.

This can be accomplished by different methods (Berendschot T and Norren D v, Arch Biochem Biophys 2004, 430:149-155, Trieschmann et al, Graefe's Arch. Clin. Exp. Opthalmol., DOI 10.1007, 2006, Springer-Verlag), for example Heterochromatic Flicker ((Delori F C, J Opt Soc Am A Opt Image Sci. Vis. 2001, 18(6):1212-30)), Scanning Laser Opthalmoscopy (SLO), Fundus Reflectometry, Raman Spectroscopy (Ermakov I, J Biomed Opt. 2004 January-February; 9(1):139-48) or U.S. Pat. No. 5,873,831 (Bernstein/University of Utah).

In a preferred embodiment of the invention, the (profile of) macular pigment optical density (MPOD) can be measured, e.g. by a technique which is based on the method described by Delori et al. (Delori F C et al., J Opt Soc Am A Opt Image Sci. Vis. 2001, 18(6):1212-30). This technique can record spatial profiles of the density of the yellow macular pigment across the retina. On a visual display, subjects view a target that alternates between two spectrally different components. One component, the blue light, is absorbed by the macular pigment, whilst the other component, which appears orange to the eye, is not absorbed by the macular pigment. This differential absorption causes an imbalance between the luminance of these two components and in turn causes the test stimulus to appear to be flickering. Flicker can be eliminated by increasing the luminance of the blue component to compensate for the absorption by the macular pigment, and the lowest luminance just required for this condition is a quantitative measure of MPOD. For the construction of MPOD profiles these measurements are done with the test target presented at different points across the retina.

The heterochromatic flicker photometry (HCF) technique uses a visual display and provides a rapid and convenient macular assessment profile (MAP) test. The MAP test is based on the use of an optical notch filter to separate the outputs of the three phosphors of the display into two components, one that is absorbed maximally by the MP and is derived only from the blue gun (i.e., the test beam) and the other that is based on a combination of red and green phosphor luminances and consists largely of long wavelength light that is not absorbed by the MP (i.e., the reference beam). The luminance of the reference beam is 20 cd m−2 and its modulation depth is fixed at 20%. The MAP test makes full use of the advantages of visual displays to produce stimuli of varying size at a number of randomised locations, to generate counter-phased sinusoidal modulation of the two stimulus beams. The frame rate of the display is 140 Hz and the stimulus modulation frequency was 20 Hz. The high temporal modulation frequency employed ensures that at threshold one isolates the activity of luminance flicker detection mechanisms that rely only on the combined L and M cone signals. The stimulus is presented as a short burst of flicker of approximately 0.5 s duration and the subject's task was to report the presence or the absence of perceived flicker.

A modified staircase procedure with variable step sizes was then used to measure the mean luminance of the test beam needed to cancel the perception of flicker generated by the reference beam. The MAP test can be used to measure MPOD along any meridian at a number of specified locations from −8° to +8° eccentricity of the visual field. The test stimulus changes from a disc of 0.36° diameter, when presented at the fovea, to a sector annulus when presented at one of five discrete locations on either side of fixation across the horizontal meridian: ±8°, ±6°, ±4°, ±2.5°, ±1.25°, 0°. The width of the test annulus also increases systematically with eccentricity to facilitate the detection and the nulling of luminance flicker. A central spot and radial guides are used to help the subject maintain steady fixation. Five, randomly interleaved, repeat measurements were taken at each spatial location investigated. The test was performed at a viewing distance of 0.7 m and the stimulus was presented only to the right eye. Similar measurements made with the left eye confirmed previous findings which show good correlation in MPOD values between the two eyes.

An optical density of the macular pigment of lower than 0.2 as determined by HCF can be regarded as evidence for an existing risk of developing AMD or the existence of a macular degeneration. This may lead to the administration of a carotenoid, such as lutein or zeaxanthin, and/or one of the antioxidants or mixture thereof as defined earlier according to step (b) of the method of the present invention (Aleman T S, Duncan J L, Bieber M L, et al. Macular pigment and lutein supplementation in retinitis pigmentosa and Usher syndrome; Invest. Opthalmol. Vis. Sci. 2001; 42(8): 1873-81; Curran-Celentano J, Hammond B R, Ciulla T A, et al. Relation between dietary intake, serum concentrations, and retinal concentrations of lutein and zeaxanthin in adults in a Midwest population, Am J Clin Nutr 2001; 74(6):796-802; Koh H H, Murray I J, Nolan D, et al. Plasma and macular responses to lutein supplement in subjects with and without age-related maculopathy: a pilot study. Exp. Eye Res. 2004; 79(1):21-27).

In still another embodiment of the present invention the identification of the individual risk of a subject of developing AMD or suffering from known or undetected AMD is accomplished by determining the xanthophyll and/or carotenoid level in a body fluid, such as blood or plasma, and/or skin. The xanthophyll and carotenoid level in plasma and/or skin can be determined by methods known in the art. For example, blood (approx. 10 to 15 ml) is collected into pre-cooled Monovettes containing EDTA, and plasma prepared by centrifugation. The preparation of plasma has to be done under appropriate shielding from light. After collection, plasma samples will be stored at −35° C. in the dark. Analyses of xanthophylls and carotenoids is performed by high pressure liquid chromatography according to published protocols (Hartmann D, et al., Am J Clin Nutr 2004; 79:410-7, Aebischer C P, et al., Methods Enzymol 1999; 299:348-62.)

Xanthophyll and carotenoid plasma levels below 0.25 μmol/L, measured as described above, may be regarded as indicative for an existing risk of developing AMD or of the existence of a macular degeneration, which may require the (or benefit from) administration of lutein or zeaxanthin to the individual.

### Sampling

The determination may comprise taking (a biological) example from the individual, such as a body fluid (such as urine, saliva or blood) that may, or may not, contain cells. Preferably, a sample may comprise buccal and/or skin cells, for example taken from the mouth using for example a swab.

The genetic analysis may be performed using a microarray (one or more genes on a chip) or a multiwell plate, for example in a laboratory. It may thus involve the use of a gene/DNA chip, or a strip or other solid surface comprising one or more genes.

### Communication of Substance

The nature or identity of the substance can be communicated either to the individual, or their doctor, optician, physician, guardian, dietician or (genetic) counselor. The communication may be electronically, for example via a computer (a PC or a laptop), portable computer or mobile phone or using the internet. Alternatively, it may be communicated on paper, for example in a booklet or information pack.

The communication of the nature or identity of the substance may be provided through a handheld, pocket or bracelet, watch-type device, personal computer, a personal digital assistance (PDA), a device which may be attached to or integral with a shopping cart or trolley, a terminal to an on-line service (e.g. in an outlet or retail store, such as a super/hypermarket), for example through the internet, a telephone with voice communication, kiosk or centralised computer system.

### Genetic Determination of Susceptibility

The identification or determination of the risk of an individual may have been undetected, or indeed an increase or decrease to risk may not been known to that individual before the determination. It can be accomplished by genome analysis or, preferably, by determining the presence (or absence) of a gene polymorphism, for example involved in the development of age-related macular degeneration (or age-related macular degeneration-related disorders). The presence of a polymorphism in genes can be determined by methods known in the art. In general, this will usually involve the extraction of genomic DNA by standard procedures, for example from blood cells, or buccal mucosa cells, hair cells or any other DNA containing tissue, which is suitably easily, and usually non-invasively, accessible. Alternatively, commercially available DNA extraction kits can be employed.

The characterisation of a given genotype, which may relate to the determination of variance of polymorphisms, can be performed according to standard procedures. This technology can involve the use of DNA sequencing apparatus, for example using a 96-channel capillary sequencer, a single strand confirmation analysis using non-denaturing gel electrophoresis, denaturing gradient gel electrophoresis (using the partial melting behaviour of double stranded DNA), heteroduplex analysis involving denaturing HPLC, chemical or enzymatic cleavage of mismatch pairing method and/or mutation detection by coupled transcription-translation (protein truncation test) procedures, for example involving non-sense. The most recent techniques are real-time PCR methods like TaqMan mass spectrometry involving a single-stranded PCR fragments, for example generated by the dideoxy-nucleotide PCR methodology, DNA microarray technology for detecting SNPs, as provided by commercial suppliers (such as Affymetrix Inc., Santa Clara, Calif., USA).

### Substances and Compositions to be Provided to the Individual

The substance or composition may comprise a compound, such as an active ingredient, a drug, pharmaceutical or nutraceutical. The substance may be edible and/or comprise a food, foodstuff or feed, for example a (dietary) supplement, or pharmaceutical composition.

The substance (or composition) may be in any form, for example suitable for oral administration, such as in solid form such as tablets, including effervescent tablets, soft or hard-shell capsules, or in liquid form such as solutions or suspensions, such as an oily suspension. Besides any active ingredient, the preparation may contain one or more conventional (eg pharmaceutical) carrier materials, additives and adjuvants, for example, including one or more of gelatine, vegetable gum, sugar, vegetable oil, polyalkylene glycol, flavouring agent, preservative, stabilizer, a emulsifying agent and/or a buffer. The substance, if medicament, can be a controlled (or delayed) release formulation.

The (therapeutic) substance may be administered in various manners such as orally, intracranially, intravenously, intramuscularly, intraperitoneally, intranasally, intradermally, and subcutaneously. The pharmaceutical compositions that contain the therapeutic agent will normally be formulated with an appropriate pharmaceutically acceptable carrier or diluent depending upon the particular mode of administration being used. For instance, parenteral formulations are usually injectable fluids that use pharmaceutically and physiologically acceptable fluids such as physiological saline, balanced salt solutions, or the like as a vehicle. Oral formulations, on the other hand, may be solids, for example tablets or capsules, or liquid solutions or suspensions. In a preferred embodiment, the therapeutic agent is administered to the individual in their diet, for example in a drink or food.

The present invention may thus provide an optimisation of diet and or nutritional supplementation and or pharmaceutical administration, based on the determination of susceptibility to the relevant disorder. The optimisation, for example of nutrition or nutritional supplementation, may be for a group of individuals, usually related ones, such as a family.

If the substance is a nutritional supplement, this may include foods, capsules, pills, powders, gums and liquids or other oral dosage forms. Also encompassed are nutritional supplements that can be delivered for example to the digestive system, or intravenously, as well as supplements that can be administered through other routes, such as mucous membranes. The individual supplements may comprise excipients, impurities or other components other than the substance of interest.

Once the individual's susceptibility has been determined, one can optimise the nutritional intake, in particular of the substance or composition. In this sense the identity of the substance itself, the amount, dosage and the form in which it is ingested or administered can be tailored to that individual, so that the substance is personalised for that particular individual. The result of the examination may include a proposal to reduce intake of supplement, macronutrient or foodstuff, as well as increasing or adding a substance or other nutritional substance.

Once the individual has been identified as having (a risk of developing) AMD, or is suffering from known or undetected AMD, an effective amount of a (preferably macular) carotenoid, such as lutein and/or zeaxanthin and/or the AREDS cocktail (a component thereof) can be suggested or administered. The substance can be a xanthophyll (for example, a carotenoid possessing one or more oxygen atoms, such as an —OH or hydroxy group).

An effective amount of the carotenoid can be used. Preferably this is lutein and/or zeaxanthin and/or “the AREDS cocktail” (vitamin C, vitamin E, beta carotene, zinc and/or copper, AREDS Report No. 8, Arch. Opthalmol. 2001; 119:1417-1436, referred to as “AREDS Cocktail”, also at HKJ Opthalmol. Vol. 4, Nr. 1, (2000), p. 3142) and/or one of the components of the AREDS cocktail. For the purposes of the present invention this can be e.g., within the range of from 0.001 mg per kg body weight to about 20 mg per kg body weight. More preferred is a daily dosage of about 0.01 to about 10 mg per kg body weight, and especially preferred is about 0.1 to 1.0 mg per kg body weight per day, especially for the carotenoid, e.g. lutein and/or zeaxanthin. Preferably the carotenoid, e.g. lutein and/or zeaxant lutein and/or zeaxanthin hin are administered at a dosage of from 1 or 5 to 15, 30 or 50 mg/day, such as from 8 or 10 to 12, 15 or 20 mg/day and may be present in compositions at that (daily) dosage. Preferred compositions can contain from 8 to 12 mg lutein or zeaxanthin (and preferably both within this range).

Especially preferred for vitamin C is 1 to about 10 mg per kg body weight, for beta-carotene 0.1 to about 0.3 mg per kg body weight, for vitamin E 1 IU to about 10 IU per kg body weight, for zinc 0.1 mg per kg body weight to about 1.5 mg kg body weight, and for copper 0.01 mg per kg body weight to about 0.05 mg per kg body weight. Zinc is preferably used as zinc oxide and copper as cupric oxide.

Preferable daily dosages and/or amount in an oral (e.g. daily) formulation, such as a tablet, are as follows. The formulation may comprise an antioxidant. This may be vitamin C (such as at from 200 to 800 mg, 400 to 600 mg, such as 450 to 550 mg). There may be 1, 2 or 3 antioxidants present. In addition or alternatively, another antioxidant is vitamin E. This may be present at a dosage of from 100 to 700 IU, such as from 200 to 600 IU, preferably from 300 to 500 IU. Another preferred antioxidant, instead of or in addition to vitamins C and E, is beta-carotene. Beta-carotene may be present at from 5 to 40 mg, such as from 10 or 20 to 30 or 40 mg, preferably from 13 to 18 mg. The zinc may be present as zinc oxide, and can be an amount of from 20 to 140 mg, such as from 60 to 100 mg, preferably from 70 to 90 mg. The copper may be present at from 1 to 2 mg.

The duration of the treatment can be suitably life-long, and no shorter than the above-mentioned markers would indicate or suggest that the subject involved is no longer at risk for developing AMD or still suffers from AMD. Suitably, treatment is started with an initial dosage of 0.5-1.0 mg of carotenoid (eg. xanthophyll), such as lutein and/or zeaxanthin, per kg body weight per day for 1-2 months whereupon the dosage may be lower to secure a macular pigment optical density of three times the threshold value, i.e. 0.6.

Preferably, two or more xanthophylls are present, such as a combination of lutein and zeaxanthin. In such combination these compounds are preferably used in a ratio of 0.1-1.0:1.0-0.1 parts (by weight), such as from 0.5-1.0:1.0-0.5, especially 0.9-1.1:0.9-1.1, to each other.

In accordance with the invention, the substance, such as lutein and/or zeaxanthin and/or the “AREDS Cocktail” or its individual components can be provided in any appropriate form, suitably for oral administration, e.g. as a pharmaceutical composition, or in food or beverage. The term “providing” as used herein is to be understood as denoting the act of collecting the desired active ingredients and processing them into a suitable administration form, as well as the direction for use and/or administration to the subject involved. Higher dosages and amounts can be provided to individuals who appear to be at greater risk, for example one of more polymorphisms associated or related to AMD, and so one can correlate higher dosages with greater risk (or more polymorphisms)

In still another aspect, the invention relates to the use of a (preferably macular) carotenoid, e.g. xanthophyll, such as lutein and/or zeaxanthin and/or a vitamin C, beta-carotene, vitamin E, zinc and copper or a mixture thereof in the manufacture of a medicament for the treatment and/or prevention of age-related macular degeneration (AMD) in a subject which has been identified as being at risk of developing AMD, or as suffering from AMD, especially by one of the methods (of the invention) identified above.

### Databases and Foods/Compositions

In the determining the susceptibility of an individual, one can obtain personal data, which may be obtained through automated data analysis, interview survey subjective analysis and/or laboratory testing. A database can be provided with information concerning available nutritional supplements, including contents, price and dosage form. A further database may include information, including risks and benefits, about constituency of nutritional supplements, for example information concerning the substance.

The invention can further include apparatus for formulating the substance, for example in a food or in a nutritional supplement, usually based on the determination of susceptibility. A specific formulation may then be provided or communicated to the individual, which may or may not be standard dosage form. The invention thus additionally contemplates a vending machine or point of sale dispensing machine which can formulate, or combine, pre-prepared dosage forms of nutritional supplements, based on the opposed nutritional supplementation or the substance to be taken by the individual. Where the point of sale dispensing machine is in a public location, an interface may be provided, for example a touch screen. Optionally, an individual may be interviewed, optionally in the presence of a trained professional, with the data inputted or accepted in an appropriate format. Thus, a trained professional, such as doctor, nurse, chiropractor, social worker or nutritionist may assist in the input in medical information, etc.

### Detection of Allelic Variants or SNPs

The detection of allelic variants according to the invention may comprise contacting a polynucleotide or protein of the individual with a specific binding agent for an age-related macular degeneration variant and determining whether the agent binds to the polynucleotide or protein. The binding of the agent can indicate the presence of the age-related macular degeneration variant, and lack of binding of the agent may indicate the absence of the age-related macular degeneration variant.

The method is generally carried out in vitro on a sample from the individual. The sample typically comprises a body fluid and/or cells of the individual and may, for example, be obtained using a swab, such as a mouth swab. The sample may be a blood, saliva, skin, cheek cell or hair root sample. The sample is typically processed before the method is carried out, for example DNA extraction may be carried out. The polynucleotide or protein in the sample may be cleaved either physically or chemically, for example using a suitable enzyme. In one embodiment the part of polynucleotide in the sample is copied or amplified, for example by cloning or using a PCR based method prior to detecting the allelic variant(s) or SNP(s).

In the present invention, any one or more methods may comprise determining the presence or absence of one or more age-related macular degeneration variants in the individual. The age-related macular degeneration variant is typically detected by directly determining the presence of the polymorphic sequence in a polynucleotide or protein of the individual. Such a polynucleotide is typically genomic DNA, mRNA or cDNA. The allelic variant may be detected by any suitable method such as those mentioned below.

A specific binding agent is an agent that binds with preferential or high affinity to the protein or polypeptide having the allelic variant but does not bind or binds with only low affinity to other polypeptides or proteins. The specific binding agent may be a probe or primer. The probe may be a protein (such as an antibody) or an oligonucleotide. The probe may be labelled or may be capable of being labelled indirectly. The binding of the probe to the polynucleotide or protein may be used to immobilise either the probe or the polynucleotide or protein.

Generally in the method, determination of the binding of the agent to the age-related macular degeneration variant can be carried out by determining the binding of the agent to the polynucleotide or protein from the individual. However in one embodiment the agent is also able to bind the corresponding wild-type sequence, for example by binding the nucleotides or amino acids which flank the allelic variant position, although the manner of binding to the wild-type sequence will be detectably different to the binding of a polynucleotide or protein containing the allelic variant.

The method may be based on an oligonucleotide ligation assay in which two oligonucleotide probes are used. These probes can bind to adjacent areas on the polynucleotide which contains the allelic variant, allowing after binding the two probes to be ligated together by an appropriate ligase enzyme. However the presence of single mismatch within one of the probes may disrupt binding and ligation. Thus ligated probes will only occur with a polynucleotide that contains the allelic variant, and therefore the detection of the ligated product may be used to determine the presence of the allelic variant.

In one embodiment the probe is used in a heteroduplex analysis based system. In such a system when the probe is bound to polynucleotide sequence containing the allelic variant it forms a heteroduplex at the site where the allelic variant occurs and hence does not form a double strand structure. Such a heteroduplex structure can be detected by the use of single or double strand specific enzyme. Typically the probe is an RNA probe, the heteroduplex region is cleaved using RNAase H and the allelic variant is detected by detecting the cleavage products.

The method may be based on fluorescent chemical cleavage mismatch analysis which is described for example in PCR Methods and Applications 3, 268-71 (1994) and Proc. Natl. Acad. Sci. 85, 4397-4401 (1998).

In one embodiment a PCR primer is used that primes a PCR reaction only if it binds a polynucleotide containing the allelic variant, for example a sequence- or allele-specific PCR system, and the presence of the allelic variant may be determined by the detecting the PCR product. Preferably the region of the primer which is complementary to the allelic variant is at or near the 3′ end of the primer. The presence of the allelic variant may be determined using a fluorescent dye and quenching agent-based PCR assay such as the Taqman PCR detection system. In a preferred embodiment, one or more of the probes and/or primers are used in a Taqman assay to detect an allelic variant.

The specific binding agent may be capable of specifically binding the amino acid sequence encoded by a variant sequence. For example, the agent may be an antibody or antibody fragment. The detection method may be based on an ELISA system. The method may be an RFLP based system. This can be used if the presence of the allelic variant in the polynucleotide creates or destroys a restriction site that is recognised by a restriction enzyme.

The presence of the allelic variant may be determined based on the change which the presence of the allelic variant makes to the mobility of the polynucleotide or protein during gel electrophoresis. In the case of a polynucleotide single-stranded conformation allelic variant (SSCP) or denaturing gradient gel electrophoresis (DDGE) analysis may be used. In another method of detecting the allelic variant a polynucleotide comprising the polymorphic region is sequenced across the region which contains the allelic variant to determine the presence of the allelic variant.

### Detection Kit

The invention also provides a kit that comprises means for determining the presence or absence of one or more age-related macular degeneration allelic variant(s) in an individual. In particular, such means may include a specific binding agent, probe, primer, pair or combination of primers, or antibody, including an antibody fragment, as defined herein which is capable of detecting or aiding detection of an age-related macular degeneration allelic variant. The primer or pair or combination of primers may be sequence specific primers which only cause PCR amplification of a polynucleotide sequence comprising the age-related macular degeneration variant to be detected, as discussed herein. The kit may also comprise a specific binding agent, probe, primer, pair or combination of primers, or antibody which is capable of detecting the absence of the allelic variant. The kit may further comprise buffers or aqueous solutions.

The kit may additionally comprise one or more other reagents or instruments which enable any of the embodiments of the method mentioned above to be carried out. Such reagents or instruments may include one or more of the following: a means to detect the binding of the agent to the allelic variant, a detectable label such as a fluorescent label, an enzyme able to act on a polynucleotide, typically a polymerase, restriction enzyme, ligase, RNAse H or an enzyme which can attach a label to a polynucleotide, suitable buffer(s) or aqueous solutions for enzyme reagents, PCR primers which bind to regions flanking the allelic variant, a positive and/or negative control, a gel electrophoresis apparatus, a means to isolate DNA from sample, a means to obtain a sample from the individual, such as swab or an instrument comprising a needle, or a support comprising wells on which detection reactions can be carried out. The kit may be, or include, an array such as a polynucleotide array comprising the specific binding agent, preferably a probe, of the invention. The kit may additionally comprise a substance (or composition) for administration to the individual, as discussed before. The kit typically includes a set of instructions for using the kit.

### Screening for (Therapeutic) Substances

In one embodiment the invention provides a method for identifying a substance useful for the treatment of age-related macular degeneration, which method comprises contacting a variant age-related macular degeneration polypeptide or a polynucleotide with a test agent and determining whether the agent is capable of binding to the polypeptide or modulating the activity or expression of the polypeptide or polynucleotide. Any suitable binding assay format can be used to determine whether the age-related macular degeneration variant binds the test agent, such as the formats discussed below.

The method may be carried out in vitro, either inside or outside a cell, or in vivo. In one embodiment the method is carried out on a cell, cell culture or cell extract that comprises a variant age-related macular degeneration protein or polynucleotide. The cell may be any suitable cell, and is typically a cell in which the product is naturally expressed.

The term “modulate” includes any of the ways mentioned herein in which the agent is able to modulate activity of an age-related macular degeneration variant polypeptide or polynucleotide. This may be determined by contacting the polypeptide or polynucleotide with the test agent under conditions that permit activity of the polypeptide or polynucleotide, and determining whether the test agent is able to modulate the activity of the polypeptide or polynucleotide.

In one aspect of the invention, the test agent is a food ingredient. Hence, the invention relates to a method of screening food ingredients to determine whether they contribute to or aggravate age-related macular degeneration in susceptible individuals, or if they prevent or alleviate age-related macular degeneration.

The present invention also provides an agent identified by a screening method of the invention. An agent identified in the screening method of the invention may be used in the therapeutic treatment of a age-related macular degeneration. Such an agent may be formulated and administered in any means or amounts as discussed below.

Customised Composition (eg. Food)

In one aspect, the invention relates to a customised diet for an individual that is susceptible to age-related macular degeneration (or an age-related macular degeneration-related disorder).

Accordingly, the present invention enables the preparation of a customised composition (or diet) suitable for an individual which is susceptible to age-related macular degeneration (or an age-related macular degeneration-related disorder), wherein the customised composition or diet comprises one or more ingredient(s) that can prevent or alleviate age-related macular degeneration (or age-related macular degeneration-related disorders) and/or does not comprise components that contribute to or aggravate age-related macular degeneration (or age-related macular degeneration-related disorders). Such ingredients may be any of those known in the art to prevent or alleviate age-related macular degeneration. Alternatively, screening methods as discussed herein may identify such ingredients. The preparation of customised food may be carried out using electronic means, for example by using a computer system.

In one embodiment, the composition may be formulated to alter the profile of food proteins in order to minimise the potential for secondary dietary sensitivity. The customised food may be hypoallergenic and/or may exclude ingredients that are poorly tolerated or cause allergies, for example gluten-containing grains such as wheat, particular protein sources such as animal proteins, milk (lactose), eggs, soy, peanuts, shellfish, fruits or tree nuts.

In another embodiment, the (customised) composition may be formulated to include functional or active ingredients that help prevent or alleviate age-related macular degeneration (or an age-related macular degeneration-related disorder).

The present invention also relates to a method of providing a composition (eg. Food) suitable for an individual which is susceptible to age-related macular degeneration (or an age-related macular degeneration-related disorder) to the individual, wherein the individual has been determined to be susceptible to age-related macular degeneration (or an age-related macular degeneration-related disorder) such as by a method of the invention.

The customised composition can be made to an inventory and supplied from inventory, i.e. is pre-manufactured rather than being made to order. Therefore the composition may not be specifically designed for one particular individual but may be suitable for a relative of the individual that may also be susceptible to age-related macular degeneration (or an age-related macular degeneration-related disorder). Alternatively, the composition may be suitable for a group of individuals that are susceptible to an age-related macular degeneration-related disorder, such members of a family. In preferred embodiment, the composition is personalised or customised to meet the nutritional requirements of a specific individual.

### Bioinformatics

The sequences of the age-related macular degeneration variants or SNPs may be stored in an electronic format, for example in a computer database. Accordingly, the invention provides a database comprising information relating to age-related macular degeneration allelic variant sequences, which may include further information about the allelic variant, for example the level of association of the allelic variant with an age-related macular degeneration-related disorder or the frequency of the allelic variant in the population. The database can comprise information regarding the substance(s), which are suitable and/or not suitable, for individuals (e.g. who may possess a particular allelic variant of age-related macular degeneration).

A database may be used to determine the susceptibility of an individual to age-related macular degeneration (or an age-related macular degeneration-related disorder). Such a determination may be carried out by electronic means, for example by using a computer system (such as a PC). Typically, the determination will be carried out by inputting genetic data from the individual to a computer system; comparing the genetic data to a database comprising information relating to age-related macular degeneration allelic variants; and on the basis of this comparison, determining the susceptibility of the individual to an age-related macular degeneration-related disorder.

The invention also provides a computer program comprising program code means for performing all the steps of a method of the invention when said program is run on a computer. Also provided is a computer program product comprising program code means stored on a computer readable medium for performing a method of the invention when said program is run on a computer. A computer program product comprising program code means on a carrier wave that, when executed on a computer system, instruct the computer system to perform a method of the invention is additionally provided.

The invention also provides an apparatus arranged to perform a method according to the invention. The apparatus typically comprises a computer system, such as a PC.

In one embodiment, the computer system comprises: means for receiving genetic data from the individual; a module for comparing the data with a database comprising information relating to age-related macular degeneration allelic variants; and means for determining on the basis of said comparison the susceptibility of the individual to an age-related macular degeneration-related disorder.

### Composition/Food Manufacturing

In one embodiment of the invention, the manufacture of a customised composition may be controlled electronically. Typically, information relating to the age-related macular degeneration allelic variant(s) present in an individual may be processed electronically to generate a customised composition. The customised composition may then be used to generate electronic manufacturing instructions to control the operation of composition manufacturing apparatus.

The apparatus used to carry out these steps will typically comprise a computer system, such as a PC, which comprises means for processing the nutritional information to generate a customised composition; means for generating electronic manufacturing instructions to control the operation of composition manufacturing apparatus; and a composition manufacturing apparatus.

The composition manufacturing apparatus may comprise a packaging apparatus. The packaging apparatus typically packages the composition into a container (such as a plastic or paper bag or box). The apparatus may also comprise means for labelling the composition, typically after packaging. The label may provide information such as: ingredient list; nutritional information; date of manufacture; best before date; weight; and types of individual(s) for which the composition is suitable.

Preferred features and/or characteristics of one aspect of the invention are applicable to another aspect mutatis mutandis.

The invention is illustrated further by the Examples given below:

### Example 1

From a adult individual a cheek swab using a fibre brush or a Q-tip is taken. The cheek swab is stored at 4° C. until analyses. The buccal mucosa cells derived from this swab are used for DNA analysis and determination of the genotype. DNA extraction is performed according to commercial suppliers (e.g. Qiagen Ltd, 8634 Hombrechtikon, Switzerland) using a standardized protocols e.g. “Isolation of DNA from buccal cells using the EZI DNA Tissue Kit (Qiagen Ltd, 8634 Hombrechtikon, Switzerland)”. This protocol is designed for the isolation of total genomic and mitochondrial DNA from buccal cells. The genotype analysis can be performed involving diverse technologies which are known to a skilled person and which are available through commercial services.

One or more of the following haplotypes for risk factors would be included in the analyses:
complement factor H SNP rs1061170, known as coding variant Y402H,
angiotensin-converting enzyme lacking Alu repeat
short-chain collagen CTRP-5 polymorphism at Ser163Arg position
for ABCR mutations ABCA4 and ABCA1
paraoxonase (PON) genotype Gln192Arg and/or Met54Leu apolipoprotein E (APOE) variants epsilon-2 allele and the epsilon 4-allele pigment-epithelium-derived factor (PEDF) allele Met72Thr in exon 3
CX3CR1, (chemokine, CX3CL1) receptor SNPs: V249I and T280M
toll-like receptor 4 variant D299G
hepatic lipase −514C->T and hepatic lipase −480C-->T polymorphism −vascular endothelial growth factor (VEGF) polymorphisms in the VEGF upstream promoter/leader sequence SNPs −2578C/A, −1154G/A and −11154G/G, and −634G/C (previously denoted +405C/G, position relative to the transcription start site) as well as −634C/C and in the VEGF 5′-untranslated region polymorphism −460C/T
MMP-9 microsatellite (CA (13-17)) polymorphism, i.e. variants that have > or 22 CA repeats
3′ splice site mutation in the TIP3 gene, namely a single base insertion at the intron 4/exon 5 junction which converts the consensus sequence CAG to CAAG in the splice acceptor site

The identification of the haplotypes for the specific genes listed follows the procedures in the respective references cited for each gene. The risk evaluation according to step (a) of the claimed method may involve one or more of the individual methods discussed above, i.e., by genome analysis and/or determining the macular pigment optical density and/or xanthophyll and carotenoid plasma level.

### Example 2

A cheek swab was taken from an individual (adult man, aged 60, different from the individual in Example 1) and DNA extracted as described in Example 1. The DNA was analysed for the SNP rs106110, namely the coding variant for Y402H, using primers and probes designed on the basis of the sequence disclosed in Haines et al, Science 308; 419 (2005). The adult was found to have this polymorphism, and was recommended a course of zeaxanthin at a dosage of 12 mg/day, reducing to 6 mg/day after one month.

### Example 3

A 6 milliwatt 0.5 mm argon laser spot (488 or 514 nm) was aimed for 9 seconds at the fovea of a flat-mounted human retina from a human female, aged 65. Scattered laser light was collected and analysed by a commercial grating monochromator, such as a Spex Triple-mate, employing a cryogenically cooled CCD array. Calibration of signal intensity with actual carotenoid levels was achieved through the examination of human post-mortem eyes.

A strong Raman spectrum characteristic of the macular carotenoids at the fovea, superimposed on a weak fluorescence background was observed. As the laser spot was moved eccentrically from the fovea the Raman signal became progressively weaker. By the time the laser was 3 mm from the fovea, the strength of the Raman signal decreased by at least a factor of 20. The patient was prescribed a course of lutein at a dosage of 12 mg/day.

### Example 4

A 1 milliwatt or lower power monochromatic laser light in the 450 to 550 nm range was directed to a subject's (male, aged 55) macular area for several seconds at a spot size 1 mm. The light scattered from the macular area was then collected via an optical fibre and routed to a spectrally selective system, which filtered out the Rayleigh scattered light and selects the Raman scattered light. A light detection system then scanned and measured the intensity of the Raman shifted light at the frequencies characteristic of macular carotenoids, from approximately 1160 to 1520 cm. The subject was given a course of lutein and zeaxanthin, both at dosages of 10 mg/day.

### Examples 5 and 6

Soft gelatin capsules to be administered according to an individual was prepared comprising the following ingredients:

| Ingredient | Amount per Capsule |
|---|---|
| Lutein | 8 and 10 mg |
| Lecithin | 50 mg |
| Soy bean oil | 200 mg |

One or more capsules may be taken, suitably with breakfast.

### Examples 7 and 8

Soft gelatin capsules were prepared comprising the following ingredients:

| Ingredient | Amount per Capsule |
|---|---|
| Lutein | 8 and 10 mg |
| Zeaxanthin | 8 and 10 mg |
| Lecithin | 50 mg |
| Soy bean oil | 200 mg |

### Example 9

Soft gelatin capsules were prepared comprising the following ingredients:

| Ingredient | Amount per Capsule | |
|---|---|---|
| Lutein | 6 | mg |
| Zeaxanthin | 6 | mg |
| Lecithin | 50 | mg |
| Soy bean oil | 200 | mg |

### Example 10

Soft gelatin capsules were prepared comprising the following ingredients:

| Ingredient | Amount per Capsule |
|---|---|
| Lutein | 12 mg |
| Lecithin | 50 mg |
| Soy bean oil | 200 mg |

### Example 11

Soft gelatin capsules was prepared comprising the following ingredients:

| Ingredient | Amount per Capsule |
|---|---|
| Zeaxanthin | 12 mg |
| Lecithin | 50 mg |
| Soy bean oil | 200 mg |

### Example 12

Soft gelatin capsules were prepared comprising the following ingredients:

| Ingredient | Amount per Capsule |
|---|---|
| Lutein | 12 mg |
| Zeaxanthin | 12 mg |
| Lecithin | 50 mg |
| Soy bean oil | 200 mg |

### Examples 13 to 19

Eight soft gelatin capsules were prepared comprising the following ingredients:

| Ingredient | Amount per capsule | | | | | | | | | | | | | |
|---|---|---|---|---|---|---|---|---|---|---|---|---|---|---|
| Lutein | 6 | mg | 6 | mg | 8 | mg | 8 | mg | 10 | mg | 12 | mg | 12 | mg |
| Zeaxanthin | 6 | mg | 6 | mg | 8 | mg | 8 | mg | 10 | mg | 12 | mg | 12 | mg |
| Vitamin C | 500 | mg | 500 | mg | 600 | mg | 700 | mg | 400 | mg | 300 | mg | 400 | mg |
| Beta-carotene | 20 | mg | 300 | mg | 15 | mg | 15 | mg | 15 | mg | 10 | mg | 10 | mg |
| Vitamin E | 400 | IU | 450 | IU | 500 | IU | 500 | mg | 500 | IU | 300 | IU | 300 | IU |
| Zinc (as zinc oxide) | 80 | mg | 60 | mg | 70 | mg | 70 | mg | 60 | mg | 50 | mg | 40 | mg |
| Copper (as cupric acid) | 2 | mg | 3 | mg | 4 | mg | 4 | mg | 3 | mg | 2 | mg | 2 | mg |
| Lecithin | 50 | mg | 60 | mg | 70 | mg | 50 | mg | 60 | mg | 70 | mg | 50 | mg |
| Soy bean oil | 200 | mg | 220 | mg | 250 | mg | 250 | mg | 200 | mg | 150 | mg | 50 | mg |

1. A method for treatment and/or prevention of age-related macular degeneration (AMD) which comprises:
(a) identifying the individual risk of a subject of developing AMD or suffering from AMD; and
(b) providing an effective amount of a carotenoid and/or vitamin C, vitamin E; beta carotene, zinc and/or copper, and/or or a mixture thereof (the AREDS Cocktail) to said subject.

2. A method of claim 1 wherein the identification of step (a) is accomplished either by genome analysis or by measuring the optical density of the macular pigment.

3. A method of claim 1 wherein the carotenoid is lutein and/or zeaxanthin.

4. A method of claim 1 wherein the identification of step (a) is accomplished by determining a xanthophyll and/or carotenoid level in plasma and/or skin.

5. A method of claim 2 wherein the genome analysis is carried out to identify gene polymorphism encoding proteins related to AMD development.

6. A method of claim 5 wherein polymorphismus in the Complement Factor H coding gene is identified.

7. A method of claim 5 wherein polymorphismus in the angiotensin-converting enzyme lacking Alu repeat coding gene is identified as a risk factor.

8. A method of claim 5 wherein polymorphisms in the short-chain collagen CTRP-5 polymorphism at Ser163Arg position coding gene is identified as a risk factor.

9. A method of claim 5 wherein polymorphismus in the for ABCR mutations ABCA4 and ABCA1 coding gene is identified as a risk factor.

10. A method of claim 5 wherein polymorphismus in the paraoxonase (PON) genotype Gln192Arg and/or Met54Leu apolipoprotein E (APOE) variants epsilon-2 allele and the epsilon 4-allele pigment-epithelium-derived factor (PEDF) allele Met72Thr in exon 3 coding gene is identified as a risk factor.

11. A method of claim 5 wherein polymorphismus in the CX3CR1, (chemokine, CX3CL1) receptor SNPs: V2491 and T280M coding gene is identified as a risk factor.

12. A method of claim 5 wherein polymorphismus in the toll-like receptor 4 variant D299G coding gene is identified as a risk factor.

13. A method of claim 5 wherein polymorphismus in the hepatic lipase −514C->T and hepatic lipase −480C-->T polymorphism coding gene is identified as a risk factor.

14. A method of claim 5 wherein polymorphismus in the vascular endothelial growth factor (VEGF) polymorphisms in the VEGF upstream promoter/leader sequence SNPs-2578C/A, −1154G/A and −1154G/G, and −634G/C (previously denoted +405C/G, position relative to the transcription start site) as well as −634C/C and in the VEGF 5′-untranslated region polymorphism −460C/T coding gene is identified as a risk factor.

15. A method of claim 5 wherein polymorphismus in the MMP-9 microsatellite (CA (13-17)) polymorphism, i.e. variants that have > or 22 CA repeats coding gene is identified as a risk factor.

16. A method of claim 5 wherein polymorphismus in the 3′ splice site mutation in the TIMP3 gene, namely a single base insertion at the intron 4/exon 5 junction which converts the consensus sequence CAG to CAAG in the splice acceptor site coding gene is identified as a risk factor.

17. A method of claim 1 wherein the identification is accomplished by more than one of the methods defined in claims 1.

18. A method according to claim 1, wherein the carotenoid comprises a xanthophyll.

19. A method of claim 1 wherein in step (b) 0.001 mg to 20 mg, preferably 0.1 mg to 1.0 mg of carotenoid (lutein and/or zeaxanthin) are administered per kg body weight per day.

20. A method of claim 1 wherein in step (b) 0.001 mg to 20 mg, preferably 0.1 mg to 1.0 mg per kg body weight of lutein and/or zeaxanthin plus vitamin C (1 to about 10 mg per kg body weight), beta-carotene (0.1 to about 0.3 mg per kg body weight), vitamin E (1 IU to about 10 IU per kg body weight), zinc (0.1 mg per kg body weight to about 1.5 mg kg body weight), copper (0.01 mg per kg body weight to about 0.05 mg per kg body weight) are administered per day.

21. The method of a carotenoid, such as lutein and/or zeaxanthin, and/or a mixture of vitamin C, beta-carotene, vitamin E, zinc and copper in the manufacture of a medicament for the treatment and/or prevention of age-related macular degeneration (AMD) in a subject which has been identified as being at risk of developing AMD, or as suffering from AMD.

22. The method as in claim 21 wherein the risk of developing AMD, or as suffering from AMD has been determined.

23. The method as in claim 21 wherein the medicament contains an amount of carotenoid, such as lutein/and or zeaxanthin, which is sufficient to administer 0.001 mg to 20 mg, preferably 0.1 mg to 1.0 mg of lutein and/or zeaxanthin per kg body weight per day.

24. A method of determining a substance to be administered to an individual, a method comprising:
a) determining the susceptibility of the individual to age-related macular degeneration (AMD); and
b) on the basis of the determination in (a), identifying a substance capable of treating and/or preventing age-related macular degeneration in that individual.

25. A method according to claim 24, comprising
(i) conducting or performing a genome or genetic analysis of the individual;
(ii) obtaining or preparing a pharmacogenomic or metabolomic profile and/or identity based on personal and/or clinical information from or about the individual;
(iii) performing a test or assay (such as on a biological sample from the individual) for a marker (e.g a biomarker, such as a compound present in the individual's body) or a physical condition that can indicate susceptibility (to age-related macular degeneration).

26. A method according to claim 24 additionally comprising:
c) providing (such as administering or communicating) of the substance (or its identity) to the individual.

27. A method according to claim 24 when the determination of the susceptibility comprises assessing or determining the risk or predisposition to age-related macular degeneration.

28. A method according to claim 24 when the determination comprises conducting or performing a genomic or genetic analysis.

29. A method according to claim 24 when the determination comprises screening for identifying an allelic variant, polymorphism (SNP) or a genetic predisposition to age-related macular degeneration.

30. A method according to claim 24 when the determination comprises additionally obtaining relevant information from the individual, such as personal and/or clinical information.

31. A method according to claim 30 when the information comprises personal information on the lifestyle, health, nutritional status, ethnic background, diet, age, sex and/or weight of the individual.

32. A method according to claim 30 when the information comprises clinical or medical information such as current or past vitamin and/or drug regime or treatments, medical conditions and/or any allergies.

33. A method according to claim 24 when the determination comprises taking a biological sample of the individual, such as a body fluid, and/or a sample comprising cells and/or analyzing a sample for a biomarker as an indicator of age-related macular degeneration.

34. A method according to claim 33 wherein the body fluid is urine, saliva and/or blood and/or the biological sample comprises buccal cells.

35. A method according to claim 24 where in (b) a proposal, suggestion or recommendation is made to the individual concerning the substance to be administered.

36. A method according to claim 24 wherein the substance is a compound (such as drug, pharmaceutical, or nutraceutical), a foodstuff, feed or dietary supplement.

37. A method for treatment and/or prevention of age-related macular degeneration, which method comprises:
a) identifying or assessing the risk of an individual developing age-related macular degeneration (AMD); and
b) providing an effective amount of a substance to the individual, wherein the substance is able to prevent or treat age-related macular degeneration, or mitigate or alleviate symptoms of age-related macular degeneration.

38. A method of preparing a customized composition for an individual which is susceptible to AMD or an age-related macular degeneration-related disorder, the method comprising:
(a) determining whether the individual is susceptible to AMD or an age-related macular degeneration-related disorder by a method according to claim 1; and
(b) preparing a composition suitable for, or tailored to, the individual.

39. A method according to claim 38, wherein the customized composition comprises ingredients which prevent or alleviate age-related macular degeneration (or an age-related macular degeneration-related disorder) and/or does not comprise ingredients which contribute to or aggravate age-related macular degeneration (or an age-related macular degeneration-related disorder).

40. A method according to claim 38 wherein the customized composition comprises a therapeutic substance.

41. A method of providing a customized composition, the method comprising providing a composition suitable for the individual that is susceptible to age-related macular degeneration (or an age-related macular degeneration-related disorder), wherein the individual has been (eg. genetically) determined to be susceptible to age-related macular degeneration (or an age-related macular degeneration-related disorder).

42. A method for identifying a substance for the treatment of age-related macular degeneration (or an age-related macular degeneration-related disorder), the method comprising:
(a) contacting an age-related macular degeneration allelic variant polypeptide or a polynucleotide which encodes an age-related macular degeneration allelic variant with a test agent; and
(b) determining whether the agent is capable of binding to the polypeptide or modulating the activity or expression of the polypeptide or polynucleotide and providing (such as administering or communicating) the substance (or its identity) to an individual.

43. A compound which is therapeutic for age-related macular degeneration (or an age-related macular degeneration-related disorder) in the manufacture of a medicament for the prevention or treatment of age-related macular degeneration (or an age-related macular degeneration-related disorder) in an individual that has been identified as being susceptible to age-related macular degeneration (or an age-related macular degeneration-related disorder) by a method according to claim 1.

44. A method of treating an individual for age-related macular degeneration (or an age-related macular degeneration-related disorder), the method comprising administering to the individual (an effective amount of) a therapeutic compound which prevents or treats AMD or the related disorder, wherein the individual has been identified as being susceptible to an age-related macular degeneration-related disorder by a method according to claim 1.

45. A database comprising information relating to age-related macular degeneration allelic variants and optionally their association with age-related macular degeneration, or age-related macular degeneration-related disorder(s) and/or substances capable of preventing or treating age-related macular degeneration.

46. A method for determining whether an individual is susceptible to age-related macular degeneration (AMD), or an age-related macular degeneration-related disorder, the method comprising:
(a) inputting data of one or more age-related macular degeneration allelic variant(s) present in the individual to a computer system;
(b) comparing the data to a computer database, which database comprises information relating to age-related macular degeneration allelic variants and the age-related macular degeneration susceptibility associated with the variants; and
(c) determining on the basis of the comparison whether the individual is susceptible to age-related macular degeneration (or an age-related macular degeneration-related disorder).

47. A computer program comprising program code means for performing all the steps of claim 46 when said program is run on a computer.

48. A computer program product comprising program code means stored on a computer readable medium for performing the method of claim 24 when said program product is run on a computer.

49. A computer program product comprising program code means on a carrier wave, which program code means, when executed on a computer system, instruct the computer system to perform a method according to claim 48.

50. A computer system arranged to perform a method according to claim 46 comprising:
(a) means for receiving data of the one or more age-related macular degeneration allelic variant(s) present in the individual;
(b) a module for comparing the data with a database comprising information relating to age-related macular degeneration allelic variants and the age-related macular degeneration susceptibility associated with the variants; and
(c) means for determining on the basis of said comparison whether the individual is susceptible to age-related macular degeneration, or an age-related macular degeneration-related disorder.

51. A method of preparing a customized composition for an individual which is susceptible to age-related macular degeneration, or an age-related macular degeneration-related disorder, the method comprising:
(a) determining whether the individual is susceptible to an age-related macular degeneration-related disorder by a method according to claim 46;
(b) (eg. electronically) generating a customized composition suitable for the individual;
(c) optionally, generating electronic manufacturing instructions to control the operation of composition manufacturing apparatus in accordance with the customized composition; and
(d) manufacturing the customized composition (according to the electronic manufacturing instructions).

52. A computer system according to claim 51, further comprising:
(e) means for electronically generating a customized composition formulation suitable for the individual;
(f) means for generating electronic manufacturing instructions to control the operation of composition manufacturing apparatus in accordance with the customized composition; and (g) a composition product manufacturing apparatus.

53. A computer system as defined in claim 52 to make a customized composition.
